(19) United States
(12) Patent Application Publication
(10) Pub. No.: US 2023/0165806 A1
(43) Pub. Date: Jun. 1, 2023

(54) Topical Therapy of Skin Fibrosis and Melanoma

(71) Applicant: Board of Regents, The University of Texas System, Austin, TX (US)

(72) Inventors: Md Nurunnabi, El Paso, TX (US); Md Nurul Huda, El Paso, TX (US)

(21) Appl. No.: 18/057,554

(22) Filed: Nov. 21, 2022

Related U.S. Application Data
(60) Provisional application No. 63/264,295, filed on Nov. 19, 2021.

(51) Int. Cl. A61K 9/70; A61K 31/635; A61P 17/02

(52) U.S. Cl. CPC A61K 9/7023; A61K 31/635; A61P 17/02; A61K 9/0014

(57) ABSTRACT

Procedures for topical treatment of melanoma and skin fibrosis include administering to a mammal in need of treatment a compound including at least one BCL-2/BCL-xl inhibitor; and at least one ionic liquid. The compound can include Navitoclax dissolved in choline octanate in a concentration from approximately 150 ug/mL to approximately 160 ug/mL.

### CROSS REFERENCE TO RELATED APPLICATION

This application is a utility conversion and claims priority to U.S. Ser. No. 63/264,295, filed Nov. 19, 2021, the contents of which are incorporated herein by reference in their entirety for all purposes.

### BACKGROUND INFORMATION

### 1. Field

The present invention relates generally to the field of medicine and disease treatment. More particularly, it concerns methods and compositions for topical therapy of melanoma and skin fibrosis.

### 2. Background

The skin is the first line of defense of the human body system against microorganisms, ultraviolet exposure, and toxic chemicals. Therefore, proper wound healing is a crucial part of skin tissue damage. Skin is made of three layers, epidermis, which create waterproof barrier and tone of our skin. The dermis comprises connective tissues, sweat glands, hair foliage, and the hypodermis layer forms fat and connective tissues. Self-repair is beneficial, but abnormal repair triggers fibrosis, malfunctioning skin function, and impairing appearance. About 30,000 people in the USA have skin fibrosis. The disease affects about 1 in 2,500 to 3,500 Caucasian newborns. It is also common in southern border regions of the USA. It affects about 1 in 100,000 Asian-Americans and 1 in 17,000 African-Americans. In most cases, skin fibrosis develops due to chemotherapeutic, radiation exposure, aberrant injury, environmental pollution, and genetic mutation. The skin fibrosis diagnosis is very impenetrable and visible in the later stage of the disease. One of the traditional methods to diagnose skin fibrosis is using skin biopsy. Current treatments for skin fibrosis consist of medication and surgery. But most treatments are only improving the fibrotic condition. The topical treatment is administered to enhance the inflammation, itching, burning, and pain due to fibrosis. Laser surgery is available to pierce and drain the fibrotic skin regions to relieve pain. Therefore, we need an alternate approach to reverse the fibrotic cells into healthy cells. Before identifying any solution, let’s discuss the physiology and signaling pathways of disease.

Fibrosis usually accompanies chronic inflammation and an increase of fibrous connective tissues in the dermis or subcutis. This condition characterizes the proliferation of fibroblasts and collagen fibers in the dermis or surrounding hair follicles, usually parallel to the epidermis. Fibrosis can develop further into the dermis and subcutis in more severe cases. Compared to the non-fibrotic dermis, areas of fibrosis may appear slightly basophilic; however, this might vary depending on the fibrotic lesion’s staining quality and development. Fibroblasts are generally accompanied by inflammatory cells in early fibrosis (primarily when associated with an epidermal ulcer); fibroblasts are larger and more active, and collagen fibers are less compact and disordered. The fibroblasts become smaller, and more spindle shaped as fibrosis progresses, the collagen fibers become more ordered and compact, and inflammation decreases. In difficult situations, hair follicles in the fibrotic region may be lost. The microvasculature (endothelial cells, platelets, capillaries) system is affected, and the disease is known as “Raynaud’s phenomenon”. Typically, skin fibroblasts synthesize little extracellular matrix (ECM) due to negative signaling and inhibitory influence of the non-cellular matrix. But due to the over-activation of fibroblast, different growth factors like IL-1, prostaglandin E, PDGF, connective tissue growth factor (CTGF), IL-6, and TGF-β. This upstream activation triggers the release of extracellular matrix and accumulates in fibroblast cells’ outer or cytoplasmic regions. In fibrosis, the cells become more rigid, lose their functionality, and apoptosis incidence stops. And eventually, all these tissue abnormality leads to organ failure.

The American Cancer Society estimates that over 106,000 new cases of skin melanoma will be diagnosed in 2021 in the United States. Estimated mortality will exceed 7100 Americans in 2021, and the mortality rate is expected to continue to increase rapidly over the next few decades. Among Caucasian people, the incidence of melanoma will grow 3-5%, making it fastest-growing cancer worldwide. There are many risk factors for melanoma, including oncogenes, smoking, and excessive exposure to the sun. Besides, some oncogenes that change mole (common skin growth) or normal skin region are responsible for melanoma. Most common oncogenes are BRAF, NRAS, CDKN2A, and NF1 are responsible for melanoma. Despite the devastating health effects of skin melanoma, current treatments include surgical excision, chemotherapy, electrodesiccation or Mohs surgery, curettage, and cryotherapy. However, unfortunately, these treatments are highly effective only against more localized cancer, rather than multiple lesions or those located at anatomically sensitive skin regions. For instance, topical gel or creams currently effectively treat the skin diseases located on superficial areas, but not the deeper skin layer where many other lesions manifest. Additional limitations include that some topical products like hydroquinone formula, fluorouracil, and Efudex create inflammation, burning, stinging, swelling when applied to the skin, which decreases patient compliance.

Heretofore, the requirements of ameliorating skin fibrosis and melanoma while avoiding side effects and other undesirable consequences have not been fully met. In view of the foregoing, there is a need in the art for a solution that simultaneously solves all of these problems.

### SUMMARY

An overall goal of embodiments of the present disclosure is to provide an ionic liquid mediated topically administered of BCL/BCL-xL inhibitor that can treat skin fibrosis. Another overall goal of embodiments of this disclosure is to provide Topical Delivery of BCL-2 Inhibitor that can treat early-stage skin melanoma.

Pathological fibrosis is distinguished from physiological wound healing by persistent myofibroblast activation, suggesting that therapies that induce myofibroblast apoptosis selectively could prevent progression and potentially reverse establish fibrosis for instances scleroderma (a heterogeneous autoimmune disease characterized by multi-organ fibrosis). Navitoclax (NAVI) is a BCl-2 and BCL-xL inhibitor that has anti-fibrotic properties and has been investigated for skin fibrosis study. NAVI, a “BH3 mimic” medication that inhibits BCL-xL, makes myofibroblast particularly vulnerable to apoptosis. BCL-xL binding to BIM is displaced by NAVI, allowing BIM to initiate apoptosis in stiffness-primed myofibroblasts. Despite of significant potency and potential application of NAVI has been controlled and limited due to their tremendous thrombocytopenia toxicity. Therefore, we have utilized a newly developed ionic liquid Topical delivery of therapy to treat skin-related disease face significant challenges. Subcutaneous layer act as the major obstacle for transporting drugs, pathogen, and viral particles. And is no study based on the topical delivery of NAVI to treat skin fibrosis. Ionic liquid composed of octanoic acid and choline bicarbonate (COA) has intrinsic properties of increasing skin pore size and allowing its payload to cross the barrier. Death signals such as the pro-apoptotic BH3-only protein BIM prime mitochondria in activated myofibroblast, but not in quiescent fibroblasts, upregulate expression of the anti-apoptotic protein BCL-xL to sequester BIM and ensure myofibroblast survival. By causing myofibroblast death, therapeutic inhibition of BCL-xL with NAVI efficiently cures preexisting fibrosis in a mouse model of scleroderma cutaneous fibrosis. Together our findings elucidate that topical delivery of COA/NAVI targeting myofibroblast anti-apoptotic proteins with BH3 mimetic drugs in scleroderma inhibits fibrotic markers in mouse disease models and improves disease condition in the animal model. Additionally, the was improved via topical delivery NAVI along with COA.

Skin melanoma remains one of the fastest-growing and the most common types of cancer in the United States and worldwide. Although primary melanoma can be removed by surgery if detected, advanced metastatic melanoma cannot be managed by surgery alone and thus an alternative and effective therapeutic approach is required. Direct local application of anti-cancer drugs may effectively treat topical cancerous skin lesions when surgery is not feasible. However, these therapies are severely limited due to the protective nature of the skin and the fact that current topical treatments may cause skin irritation, burning, light sensitivity, scaling, and inflammation. To treat early-stage melanoma, we have developed a topical formulation of Navitoclax (NAVI), a BCL-2 inhibitor that upregulates apoptosis, in association with an ionic liquid composed of Choline Octanoate (COA) at a molar ratio of 1:1. NAVI is a small hydrophobic molecule that solubilizes at 20% (w/v) when dissolved into 50% (DW v/v) COA. NAVI is a highly effective chemotherapeutic yet equally hemotoxic. Therefore, COA-mediated topical delivery of NAVI enhanced transportation through the skin barrier and held the drug in the deeper skin layers for an extended period compared to the control. Compared to oral administration of NAVI, topical administration of COA/NAVI exhibited higher cancer-cell killing specificity with less toxicity to healthy human skin cells. In vivo experiments confirm its efficacy in treating melanoma without irritation or systemic absorption. In the mouse xenograft model, the COA/NAVI formulation induced apoptosis and provided a safe and potent topical, transdermal delivery for the treatment of melanoma.

An illustrative embodiment of the present disclosure is a composition of matter for topical treatment of disease, comprising at least one BCL-2/BCL-xl inhibitor; and at least one ionic liquid. The at least one BCL-2/BCL-xl inhibitor can include at least one BCL-2 inhibitor selected from the group consisting of Navitoclax, ABT-737, Venetoclax or BM-1074; and the at least one ionic liquid can include choline octanate. The composition can be included in a transdermal patch.

Another illustrative embodiment of the present disclosure is a method of topical treatment of skin fibrosis, comprising administering to a mammal in need thereof a compound comprising at least one BCL-2/BCL-xl inhibitor; and at least one ionic liquid. The ionic liquid can be choline octanate produced by reacting choline bicarbonate and octanoic acid before administering.

Another illustrative embodiment of the present disclosure is a method of topical treatment of melanoma, comprising administering to a mammal in need thereof a compound comprising at least one BCL-2/BCL-xl inhibitor; and at least one ionic liquid. Preparation of the compound can include dissolving Navitoclax in choline octanate in a concentration of from approximately 150 ug/mL to approximately 160 ug/mL.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 illustrates oral gavage of NAVI and topical administration of COA/NAVI in accordance with an illustrative embodiment.

FIG. 2 illustrates blood quantification for NAVI PO and COA/NAVI in accordance with an illustrative embodiment.

FIG. 3 illustrates fibrotic area from beginning of treatment for 4 groups in accordance with an illustrative embodiment.

FIG. 4 illustrates body weight change for 4 groups in accordance with an illustrative embodiment.

FIG. 5 illustrates cell death % by apoptosis and necrosis for 8 groups in accordance with an illustrative embodiment.

FIG. 6 illustrates Western blot analysis for 5 groups in accordance with an illustrative embodiment.

FIG. 7 illustrates BCL-2 expression for 5 groups in accordance with an illustrative embodiment.

### DETAILED DESCRIPTION

The disclosure of this application is technically related to co-pending U.S. Ser. No. ______ (attorney docket number UTEP2022-005-2), filed Nov. 21, 2022, the entire contents of which are hereby expressly incorporated by reference for all purposes.

Embodiments of this disclosure include methods and formulations that will ameliorate skin fibrosis. While not being bound by theory, the methods and formulations induce apoptosis of fibrosed cells.

A topical solution-based therapy combines an ionic liquid with a BCL-2 inhibitor. The ionic liquid based on choline bicarbonate and octanoic acid (COA) has been reintroduced as a highly effective method of transporting tiny hydrophobic molecules to the skin. By inhibiting the BCL-2 protein of a specific cell, the BCL-2 inhibitor Navitoclax (NAVI) was also very successful and safe chemotherapy for treating cancer. The COA/NAVI combination was created to promote skin penetration and drug retention in the target area for a more extended period, increasing therapeutic efficiency. Despite NAVI’s developing potential as a chemotherapeutic drug, its use is restricted because of the danger of severe thrombocytopenia. As a result, the regulatory body has limited their administration route to oral rather than intravenous or intramuscular.

The main challenges in treating fibrosis with chemotherapeutics are epidermal layer penetration, the appropriate concentration, and drug retention within the layer for a more extended period. The COA ionic liquid, as created, is intended to improve NAVI skin permeation, and keep the payload within the dermis and subcutaneous layer of the skin. NAVI is an FDA-approved hydrophobic anti-apoptotic protein B cell lymphoma/leukemia (BCL-2) inhibitor, making it a good candidate for COA enhanced negation of these variables. NAVI is a BH3 mimetic drug that binds directly to BCL-2, BCL-xL, and Bcl-w. BH3 mimetics are tiny molecules that inhibit anti-apoptotic BCL-2 family members. Individual anti-apoptotic factors like BCL-2, BCL-xL, BCL-W, and MCL are blocked. COA improves NAVI skin permeability and increases its solubility by up to 150 µg/mL. With the development of COA/NAVI formulations, we overcame the clinical limitation of NAVI and facilitated its topical application to accelerate the treatment of local fibrosis. The COA/NAVI formulation delivers and maintains the drug at the desired fibrosis site for a more extended period, as confirmed by a pigskin in vitro permeation test (IVPT). In vitro studies performed on the human skin fibrotic skin fibroblast cell line Hs27 show its potential use as a chemotherapeutic agent for fibrosis treatment, and COA/NAVI shows significantly greater efficacy than free NAVI. In vivo studies were performed in a bleomycin-induced mouse model to evaluate the drugs’ effectiveness, safety, and bio distribution. The results of studies and general observations indicate that the COA/NAVI formulation can be considered an effective and safe treatment approach for treating cutaneous fibrosis.

A topical solution-based treatment comprises an ionic liquid and BCL-2 inhibitor. The Choline Octanoate (COA) ionic liquid was reported as a very effective agent for delivering small hydrophobic molecules through the skin. The BCL-2 inhibitor navitoclax (NAVI) was also a very effective and safe chemotherapeutic to treat cancer by inhibiting the BCL-2 protein of a specific cell. The COA/NAVI combination was specifically designed to improve skin penetration and enhance retention of the drug in the target area for an extended time to enhance therapeutic efficiency. Despite the emerging potential of NAVI as a chemotherapeutic agent, its applications are limited due to the risk of severe hematotoxicity. Therefore, the regulatory authority has restricted their route of administration to oral instead of intravenous and intramuscular.

The major challenges of melanoma treatment with chemotherapeutics are inadequate epidermal layer penetration, inadequate concentration, and poor drug retention within the layer for extended time periods. The as-developed ionic liquid COA is designed to enhance the skin permeation of NAVI and hold the payload within the dermis and subcutaneous layer of the skin. NAVI is a hydrophobic, anti-apoptotic protein B cell lymphoma/leukemia (BCL-2) inhibitor approved by the FDA for cancer treatment, making it an ideal candidate for COA enhancement negation of these factors. NAVI, a BH3 (BH3 mimetics are small compounds that antagonize anti-apoptotic BCL-2 family proteins) mimetic drug directly binds with BCL-2, Bcl-xL, and Bcl-w. It blocks individual anti-apoptotic factors like BCL-2, BCL-xL, BCL-W, and MCL. COA not only enhances skin permeation of NAVI but also increases solubility up to 150 µg/mL. With the development of COA/NAVI formulation, we have overcome the clinical limitation of NAVI and facilitated their topical application to accelerate local melanoma treatment. COA/NAVI formulation delivers and holds the drug for an extended period at the targeted melanoma site, confirmed by in vitro permeation test (IVPT) using porcine skin. In vitro studies conducted on A375 human melanoma cells line demonstrate their potential use as a chemotherapeutic for treating melanoma, and COA/NAVI shows significantly higher efficacy than free NAVI. In vivo studies were conducted on an A375 xenograft mouse model to evaluate drug efficacy, safety, and biodistribution. The findings from comprehensive studies and observations reveal that the COA/NAVI formulation can be an effective and safer therapeutic approach for treating skin melanoma.

The components of the formulation compound can be substituted partially or wholly with other components that provide equivalent functionality. Examples of components that can be substituted include the following.

List of BCL-2 inhibitors

| Inhibitors Name | CAS |
|---|---|
| Navitoclax | 923564-51-6 |
| ABT-737 | 852808-04-9 |
| Obatoclax Mesylate | 803712-79-0 |
| TW-37 | 877877-35-5 |
| Venetoclax | 1257044-40-8 |
| BM-1074 | 1391108-10-3 |
| Gambogic Acid | 2752-65-0 |

### List of Ionic Liquid Reagents

Choline Bicarbonate (CAS: 78-73-9),
Octanoic Acid (CAS: 124-07-2),
Octenoic Acid (CAS: 18719-24-9),
and Geranic Acid (CAS: 459-80-3)

The choline octanate can be produced by reacting at least two members selected from the group consisting of choline bicarbonate, octanoic acid, octenoic acid or geranic acid. More specifically choline bicarbonate and at least one member selected from the group consisting of octanoic acid, octenoic acid or geranic acid.

### EXAMPLES

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

### Example 1

Mice were randomly distributed into four groups and treatments were given for four weeks. Our treatment groups were untreated, NAVI (50 µM) (PO), topical administration of COA (50%), and COA/NAVI (50 µM). (a)

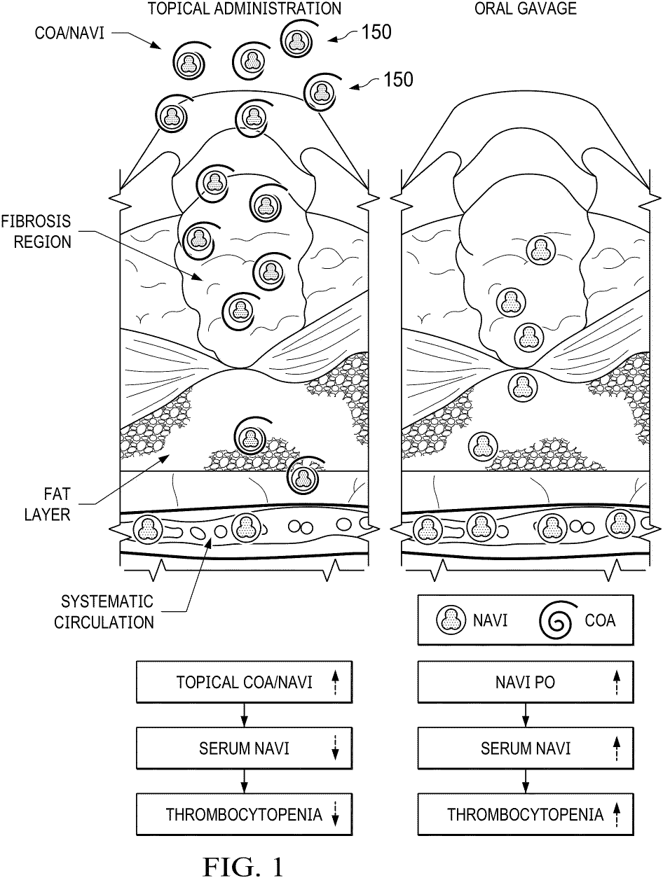

Referring to FIG. 1, COA/NAVI 150 can be seen in a schematic representation of how NAVI transport via the topical and oral route of administration. Topical COA/NAVI delivery ensures a higher percentage of NAVI in the fibrotic area than NAVI PO administration. Also, topical COA/NAVI holds the drug longer in the fibrotic region and allows less NAVI transportation in the systemic circulation. Thus, we observe lower thrombocytopenia in COA/NAVI topical administration compared to NAVI PO.

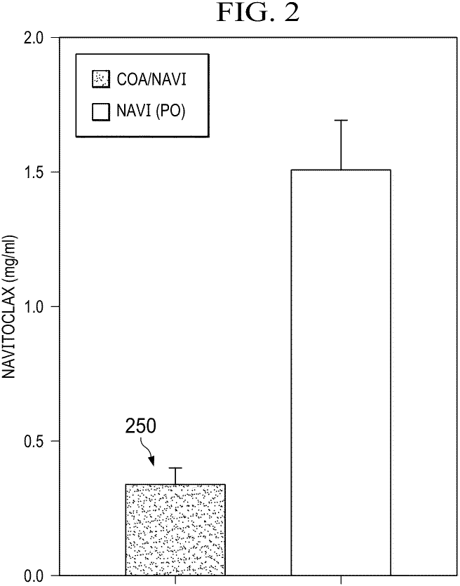

Referring to FIG. 2, blood samples were quantified using a UV plate reader at 345 nm, and NAVI concentrations were quantified. The graph shows higher transport of NAVI (1.61 mg/mL) 250 via GI track than COA/NAVI (0.38 mg/mL) via skin fat layers.

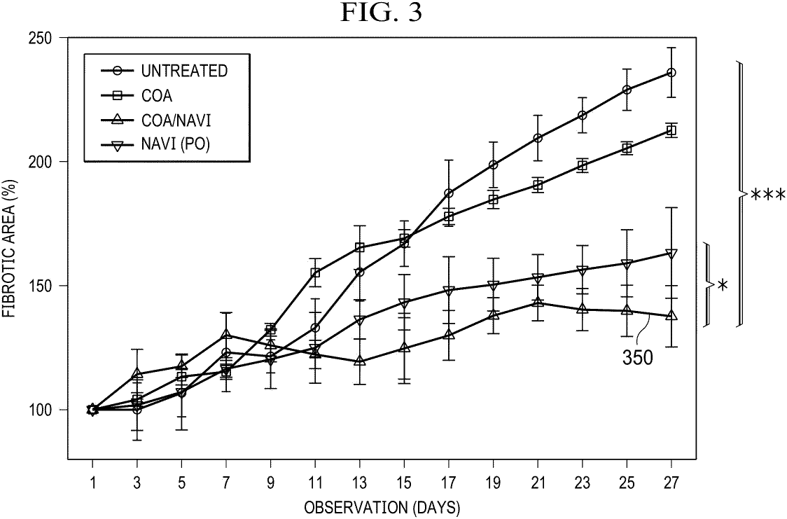

Referring to FIG. 3, the fibrotic area was measured using slide calipers from the beginning of treatment. Data for COA/NAVI 350 shows fibrotic area was inhibited by 120% compared to the untreated group after four weeks of treatment. This data validates our initial claim of COA/NAVI anti-fibrotic therapeutic efficiency.

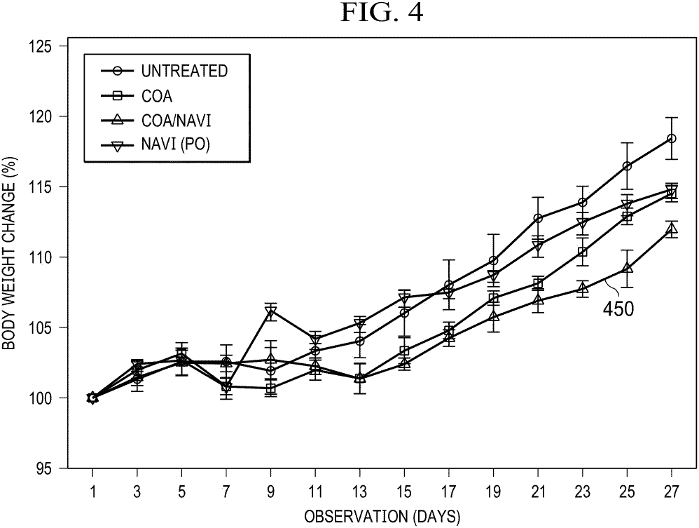

Referring to FIG. 4, body weight was measured during the treatments. We observe no sudden reduction of body weight, indicating our treatments were safe to administer to mice. The weight data for COA/NAVI 450 showed the smallest change. The data shown here is mean ± SD, the experiment was performed in N = 3. ^{∗∗∗} Indicates statistical significance with p < 0.001; ^{∗∗} indicates statistical significance with p < 0.01 and ^{∗} indicates statistical significance with p < 0.05.

### Example 2

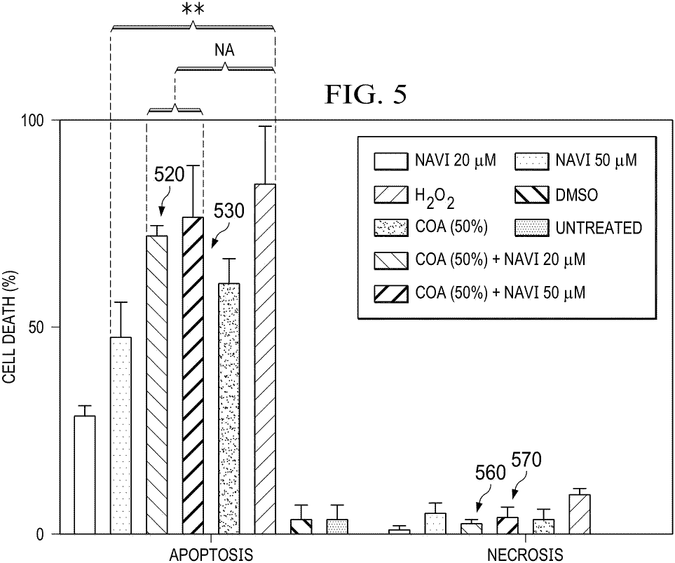

Referring to FIG. 5, in the apoptosis-necrosis assay, the formulations COA/NAVI (20 µM) 520 and COA/NAVI (50 µM) 530 showed around 70% and 75% apoptosis, respectively, tested in A375 cells after 24 h of treatment compared to H₂O₂, as a positive control. However, both treatments COA/NAVI (20 µM) 560 and COA/NAVI (50 µM) 570 showed around 5% necrosis.

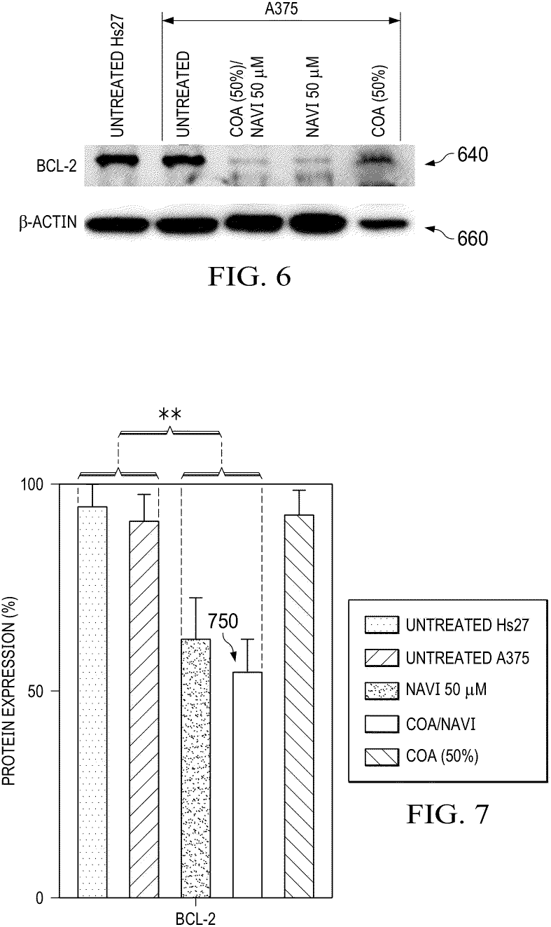

Referring to FIG. 6, Western blot analysis was done from Hs27 healthy skin fibroblast and A375 human melanoma cell lysate after treatment with the formulations. The experiment shows more inhibition of BCL-2 expression in COA/NAVI 640 and NAVI compared to COA or untreated melanoma and healthy cell. Housekeeping gene β-actin expression with COA/NAVI (50 µM) 660 is about the same.

Referring to FIG. 7, the BCL-2 expression was quantified and represented compared to housekeeping gene β-actin. BCL-2 expression in COA/NAVI 750 is lower. Data represent mean ± SD, where n=6. ^{∗} Indicates statistical significance with p < 0.05; ^{∗∗∗} indicates statistical significance with p < 0.001.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition of matter for topical treatment of disease, comprising:
at least one BCL-2/BCL-xl inhibitor; and
at least one ionic liquid.

2. The composition of claim 1, wherein the at least one BCL-2/BCL-xl inhibitor comprises at least one BCL-2 inhibitor selected from the group consisting of Navitoclax, ABT-737, Venetoclax or BM-1074.

3. The composition of claim 1, wherein the at least one BCL-2/BCL-xl inhibitor comprises at least one BCL-2 inhibitor selected from the group consisting of Navitoclax, ABT-737, Obatoclax Mesylate, TW-37, Venetoclax, BM-1074 or Gambogic Acid.

4. The composition of claim 1, wherein the at least one ionic liquid comprises choline octanate.

5. The composition of claim 4, wherein the at least one BCL-2/BCL-xl inhibitor comprises Navitoclax.

6. The composition of claim 5, wherein a concentration of Navitoclax with respect to choline octanate is from approximately 150 ug/mL to approximately 160 ug/mL.

7. The composition of claim 1, further comprising a transdermal patch.

8. A method of topical treatment of skin fibrosis, comprising:
administering to a mammal in need thereof a compound comprising
at least one BCL-2/BCL-xl inhibitor; and
at least one ionic liquid.

9. The method of claim 8, wherein administering includes administering the compound wherein the at least one BCL-2/BCL-xl inhibitor comprises at least one BCL-2 inhibitor selected from the group consisting of Navitoclax, ABT-737, Venetoclax or BM-1074.

10. The method of claim 8, wherein administering includes administering the compound wherein the at least one BCL-2/BCL-xl inhibitor comprises at least one BCL-2 inhibitor selected from the group consisting of Navitoclax, ABT-737, Obatoclax Mesylate, TW-37, Venetoclax, BM-1074 or Gambogic Acid.

11. The method of claim 8, wherein administering includes administering the compound wherein the at least one ionic liquid comprises choline octanate.

12. The method of claim 11, further comprising producing the choline octanate by reacting choline bicarbonate and octanoic acid before administering.

13. The method of claim 12, further comprising dissolving Navitoclax in choline octanate in a concentration of from approximately 150 ug/mL to approximately 160 ug/mL.

14. The method of claim 11, further comprising producing the choline octanate by reacting at least two members selected from the group consisting of choline bicarbonate, octanoic acid, octenoic acid or geranic acid before administering.

15. A method of topical treatment of melanoma, comprising:
administering to a mammal in need thereof a compound comprising
at least one BCL-2/BCL-xl inhibitor; and
at least one ionic liquid.

16. The method of claim 15, wherein administering includes administering the compound wherein the at least one BCL-2/BCL-xl inhibitor comprises at least one BCL-2 inhibitor selected from the group consisting of Navitoclax, ABT-737, Venetoclax or BM-1074.

17. The method of claim 15, wherein administering includes administering the compound wherein the at least one BCL-2/BCL-xl inhibitor comprises at least one BCL-2 inhibitor selected from the group consisting of Navitoclax, ABT-737, Obatoclax Mesylate, TW-37, Venetoclax, BM-1074 or Gambogic Acid.

18. The method of claim 15, wherein administering includes administering the compound wherein the at least one ionic liquid comprises choline octanate.

19. The method of claim 18, further comprising producing the choline octanate by reacting choline bicarbonate and octanoic acid before administering.

20. The method of claim 19, further comprising dissolving Navitoclax in choline octanate in a concentration of from approximately 150 ug/mL to approximately 160 ug/mL.

21. The method of claim 15, wherein administering includes administering the compound wherein the at least one ionic liquid comprises at least two members selected from the group consisting of choline bicarbonate, octanoic acid, octenoic acid or geranic acid.
